(19) United States
(12) Patent Application Publication
(10) Pub. No.: US 2007/0112053 A1
(43) Pub. Date: May 17, 2007

(54) Pharmaceutical compositions and methods using temozolomide and a protein kinase inhibitor

(76) Inventors: Cecil B. Pickett, Far Hills, NJ (US); Walter Robert Bishop, Pompton Plains, NJ (US); Yaolin Wang, Edison, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/522,207

(22) Filed: Sep. 15, 2006

Related U.S. Application Data
(60) Provisional application No. 60/718,011, filed on Sep. 16, 2005.

(51) Int. Cl. A61K 31/407

(52) U.S. Cl. 514/410

(57) ABSTRACT

The present invention provides formulations, kits, and methods useful for treating a cell proliferative disorder.

### CROSS REFERENCE TO RELATED APPLICATION

This application claims the benefit of priority to U.S. Provisional Patent Application No. 60/718,011, filed Sep. 16, 2005, which is hereby incorporated by reference.

### FIELD OF THE INVENTION

The present invention provides formulations, kits, and methods useful for treating a cell proliferative disorder. In particular, the formulations, kits and methods include temozolomide (TMZ) in combination with a protein kinase C (PKC) inhibitor.

### BACKGROUND OF THE INVENTION

For the year 2005, the American Cancer Society estimates the number of new cancer cases at 1,372,910 and the number of cancer related deaths at 570,280 in the United States alone. In light of the widespread number of cancer cases and cancer-related deaths, as well as the inadequacies of currently available treatments, there is a need for more effective therapeutics to treat cancer. Such cancers include glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides.

Cancer results from a defect in the regulation of processes that control cell proliferation and survival. PKC is involved in signal transduction associated with cell proliferation, differentiation, and apoptosis. In particular, PKC isozymes are involved in regulating critical cell cycle transitions, including cell cycle entry and exit and the G₁ and G₂ checkpoints. PKC isozymes are also implicated in regulation of tumor angiogenesis, the growth of new blood vessels into the tumor. PKC isozymes differ in their structure, biochemical properties, tissue distribution, subcellular localization, and substrate specificity.

Of the estimated 20,000 new brain tumors diagnosed each year in the United States, about half are malignant gliomas and result in death within 18 months. Gliomas originate from glial cells, most often astrocytes, and may occur anywhere in the brain or spinal cord, including the cerebellum, brain stem, or optic chiasm. Gliomas can be divided into two groups based on their growth characteristics: low-grade gliomas and high-grade gliomas. Low-grade gliomas are usually localized and grow slowly over a long period of time. Examples of low-grade gliomas include astrocytomas, oligodendrogliomas, pilocytic astrocytomas. Over time, most of these low-grade gliomas dedifferentiate into more malignant high-grade gliomas that grow rapidly and can easily spread through the brain. Examples of high-grade gliomas include anaplastic astrocytoma and glioblastoma multiforme.

A crucial step in continuous growth of tumors and development of metastasis is the recruitment of new blood vessels in and around tumors. A tumor mass<1 mm in diameter can receive oxygen and nutrients by diffusion, but any increase in tumor mass requires angiogenesis, Le., the proliferation and morphogenesis of vascular endothelial cells.

Recently, PKC beta has been shown to increase the formation of new blood vessels. In fact, when produced in excess, PKC beta has been shown to lead to overgrowth of new blood vessels and to increase the permeability of blood vessels resulting in increased leakage.

Despite advances in conventional therapies for malignant gliomas which include surgical removal, radiation therapy, and chemotherapy as well as combinations thereof, malignant gliomas continue to be associated with a poor prognosis. Thus, there remains a need for more effective therapeutics to treat the growth and metastasis of a variety of cancers, including gliomas.

### SUMMARY OF THE INVENTION

The present invention provides formulations, kits, and methods useful for treating a cell proliferative disorder. In one embodiment, the present invention provides formulations, kits, and methods that include TMZ or a pharmaceutically acceptable salt thereof in combination with a PKC inhibitor. In another embodiment, the present invention provides formulations, kits, and methods that include TMZ or a pharmaceutically acceptable salt thereof in combination with a selective PKC beta inhibitor. Such a combination is more effective than treatment with either therapy alone. In addition, the present formulations, kits, and methods permit a lower dose of one or more pharmaceutically active agents to be administered, than would otherwise be required, to achieve a therapeutic effect thereby reducing adverse effects associated with the dosage administered. Generally, use of a selective PKC inhibitor, more preferentially, an isozyme selective PKC inhibitor will reduce adverse side effects associated with non-selective PKC inhibitors.

In preferred embodiments, the cell proliferative disorder is glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides. In more preferred embodiments, the cell proliferative disorder is glioma, melanoma, lung cancer, lymphoma, colorectal and/or colon cancer, head and neck or ovarian cancer. In a most preferred embodiment, the cell proliferative disorder is glioma. In a preferred embodiment, the selective PKC inhibitor is UCN01 (7-hydroxystaurosporine), GÖ6976, ruboxistaurin (LY333531), N-desmethyl LY333531, LY379196, enzastaurin (LY317615), LY326020, bryostatin 1, tamoxifen, ISIS 3521 (LY900003; Affinitak; SEQ ID NO: 1), ISIS 9606 (SEQ ID NO: 2), or a pharmaceutically acceptable salt of any of these agents, or a combination of two or more of these agents. In a preferred embodiment, the present invention provides formulations comprising a therapeutically effective amount of TMZ or a pharmaceutically acceptable salt thereof in combination with a selective PKC inhibitor. In a one embodiment of the formulation, the selective PKC inhibitor is a selective PKC beta inhibitor. More preferably, the selective PKC beta inhibitor is a selective PKC beta-2 inhibitor.

In a more preferred embodiment of the formulation, the selective PKC inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the formulation, the selective PKC inhibitor is ruboxistaurin (LY333531), N-desmethyl LY333531, or a pharmaceutically acceptable salt thereof. In a more preferred embodiment of the formulation, the selective PKC inhibitor is N-desmethyl LY33353 1, or a pharmaceutically acceptable salt thereof.

In another embodiment of the formulation, the selective PKC inhibitor is enzastaurin (LY317615), LY326020, or a pharmaceutically acceptable salt thereof. In a more preferred embodiment of the formulation, the selective PKC inhibitor is LY326020, or a pharmaceutically acceptable salt thereof. In a preferred embodiment of the formulation, the pharmaceutically acceptable salt of TMZ or of the selective PKC inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the formulation, the therapeutically effective amount of TMZ or a pharmaceutically acceptable salt thereof is a standard dose intensity. In another embodiment of the formulation, the therapeutically effective amount of TMZ or a pharmaceutically acceptable salt thereof is an enhanced dose intensity.

In yet another embodiment of the formulation, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight. In another preferred embodiment of the formulation, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment, the formulation is administered daily in a 6 week cycle.

In a preferred embodiment, the present invention provides kits comprising:
a first container having a therapeutically effective amount of TMZ or a pharmaceutically acceptable salt thereof;
a second container having a therapeutically effective amount of a selective PKC inhibitor; and
instructions for use to treat a cell proliferative disorder.

In one embodiment of the kit, the selective PKC inhibitor is a selective PKC beta inhibitor. More preferably, the selective PKC beta inhibitor is a selective PKC beta-2 inhibitor.

In a more preferred embodiment of the kit, the selective PKC inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the kit, the selective PKC inhibitor is ruboxistaurin (LY333531), N-desmethyl LY333531, or a pharmaceutically acceptable salt thereof.

In a more preferred embodiment of the kit, the selective PKC inhibitor is N-desmethyl LY333531, or a pharmaceutically acceptable salt thereof.

In another embodiment of the kit, the selective PKC inhibitor is enzastaurin (LY317615), LY326020, or a pharmaceutically acceptable salt thereof.

In a more preferred embodiment of the kit, the selective PKC inhibitor is LY326020, or a pharmaceutically acceptable salt thereof.

In a preferred embodiment of the kit, the pharmaceutically acceptable salt of TMZ or of the PKC inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the kit, the therapeutically effective amount of TMZ or a pharmaceutically acceptable salt thereof is a standard dose intensity. In another embodiment of the kit, the therapeutically effective amount of TMZ or a pharmaceutically acceptable salt thereof is an enhanced dose intensity.

In yet another embodiment of the kit, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight.

In another preferred embodiment of the kit, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment of the kit, the therapeutic components are administered daily in a 6 week cycle.

In a preferred embodiment of the kit, the therapeutically effective amount of TMZ or a pharmaceutically acceptable salt thereof is administered together in time as the therapeutically effective amount of the selective PKC inhibitor.

In another preferred embodiment of the kit, the therapeutically effective amount of TMZ or a pharmaceutically acceptable salt thereof is administered separately in time as the therapeutically effective amount of the selective PKC inhibitor.

In a preferred embodiment, the present invention provides methods for treating a cell proliferative disorder in a patient suffering there from comprising administering a therapeutically effective amount of TMZ or a pharmaceutically acceptable salt thereof in combination with a selective PKC inhibitor. In a preferred embodiment of the method, the selective PKC inhibitor is a selective PKC beta inhibitor. More preferably, the selective PKC beta inhibitor is a selective PKC beta-2 inhibitor.

In a more preferred embodiment of the method, the selective PKC inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the method, the selective PKC inhibitor is ruboxistaurin (LY333531), N-desmethyl LY333531, or a pharmaceutically acceptable salt thereof. In a more preferred embodiment of the method, the selective PKC inhibitor is N-desmethyl LY333531, or a pharmaceutically acceptable salt thereof.

In another embodiment of the method, the selective PKC inhibitor is enzastaurin (LY317615), LY326020, or a pharmaceutically acceptable salt thereof. In a more preferred embodiment of the method, the selective PKC inhibitor is LY326020, or a pharmaceutically acceptable salt thereof.

In a preferred embodiment of the method, the pharmaceutically acceptable salt of TMZ or of the selective PKC inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the method, the therapeutically effective amount of TMZ or a pharmaceutically acceptable salt thereof is a standard dose intensity. In another embodiment of the method, the therapeutically effective amount of TMZ or a pharmaceutically acceptable salt thereof is an enhanced dose intensity.

In yet another embodiment of the method, the therapeutically effective amount of ruboxistaurin (LY33353 1), N-desmethyl LY33353 1, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight.

In another preferred embodiment of the method, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment of the method, the therapeutic components are administered daily in a 6 week cycle.

In one preferred embodiment, the glioma is an anaplastic astrocytoma. In another preferred embodiment, the glioma is a glioblastoma multiforme.

In a preferred embodiment, the present invention provides formulations comprising a therapeutically effective amount of TMZ or a pharmaceutically acceptable salt thereof in combination with a selective PKC beta inhibitor. In a preferred embodiment of the formulation, the selective PKC beta inhibitor is a selective PKC beta-2 inhibitor.

In a more preferred embodiment of the formulation, the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the formulation, the selective PKC beta inhibitor is ruboxistaurin (LY333531), N-desmethyl LY333531, or a pharmaceutically acceptable salt thereof. In a more preferred embodiment of the formulation, the selective PKC beta inhibitor is N-desmethyl LY33353 1, or a pharmaceutically acceptable salt thereof.

In another embodiment of the formulation, the selective PKC beta inhibitor is enzastaurin (LY317615), LY326020, or a pharmaceutically acceptable salt thereof. In a more preferred embodiment of the formulation, the selective PKC beta inhibitor is LY326020, or a pharmaceutically acceptable salt thereof. In a preferred embodiment of the formulation, the pharmaceutically acceptable salt of TMZ or of the selective PKC beta inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the formulation, the therapeutically effective amount of TMZ or a pharmaceutically acceptable salt thereof is a standard dose intensity. In another embodiment of the formulation, the therapeutically effective amount of TMZ or a pharmaceutically acceptable salt thereof is an enhanced dose intensity.

In yet another embodiment of the formulation, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight. In another preferred embodiment of the formulation, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment, the formulation is administered daily in a 6 week cycle.

In a preferred embodiment, the present invention provides kits comprising:
a first container having a therapeutically effective amount of TMZ or a pharmaceutically acceptable salt thereof;
a second container having a therapeutically effective amount of a selective PKC beta inhibitor; and
instructions for use to treat a cell proliferative disorder.

In a preferred embodiment of the kit, the selective PKC beta inhibitor is a selective PKC beta-2 inhibitor.

In a more preferred embodiment of the kit, the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the kit, the selective PKC beta inhibitor is ruboxistaurin (LY333531), N-desmethyl LY333531, or a pharmaceutically acceptable salt thereof.

In a more preferred embodiment of the kit, the selective PKC beta inhibitor is N-desmethyl LY33353 1, or a pharmaceutically acceptable salt thereof.

In another embodiment of the kit, the selective PKC beta inhibitor is enzastaurin (LY317615), LY326020, or a pharmaceutically acceptable salt thereof.

In a more preferred embodiment of the kit, the selective PKC beta inhibitor is LY326020, or a pharmaceutically acceptable salt thereof.

In a preferred embodiment of the kit, the pharmaceutically acceptable salt of TMZ or of the PKC beta inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the kit, the therapeutically effective amount of TMZ or a pharmaceutically acceptable salt thereof is a standard dose intensity. In another embodiment of the kit, the therapeutically effective amount of TMZ or a pharmaceutically acceptable salt thereof is an enhanced dose intensity.

In yet another embodiment of the kit, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight. In another preferred embodiment of the kit, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment of the kit, the therapeutic components are administered daily in a 6 week cycle.

In a preferred embodiment of the kit, the therapeutically effective amount of TMZ or a pharmaceutically acceptable salt thereof is administered together in time as the therapeutically effective amount of the selective PKC beta inhibitor.

In another preferred embodiment of the kit, the therapeutically effective amount of TMZ or a pharmaceutically acceptable salt thereof is administered separately in time as the therapeutically effective amount of the selective PKC beta inhibitor.

In a preferred embodiment, the present invention provides methods for treating a glioma in a patient suffering there from comprising administering a therapeutically effective amount of TMZ or a pharmaceutically acceptable salt thereof in combination with a selective PKC beta inhibitor. In a preferred embodiment of the method, the selective PKC beta inhibitor is a selective PKC beta-2 inhibitor.

In a more preferred embodiment of the method, the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

In one embodiment of the method, the selective PKC beta inhibitor is ruboxistaurin (LY333531), N-desmethyl LY333531, or a pharmaceutically acceptable salt thereof. In a more preferred embodiment of the method, the selective PKC beta inhibitor is N-desmethyl LY33353 1, or a pharmaceutically acceptable salt thereof.

In another embodiment of the method, the selective PKC beta inhibitor is enzastaurin (LY317615), LY326020, or a pharmaceutically acceptable salt thereof. In a more preferred embodiment of the method, the selective PKC beta inhibitor is LY326020, or a pharmaceutically acceptable salt thereof.

In a preferred embodiment of the method, the pharmaceutically acceptable salt of TMZ or of the selective PKC beta inhibitor is prepared from a pharmaceutically acceptable acid addition salt selected from the group consisting of acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluene sulfonic acid.

In one embodiment of the method, the therapeutically effective amount of TMZ or a pharmaceutically acceptable salt thereof is a standard dose intensity. In another embodiment of the method, the therapeutically effective amount of TMZ or a pharmaceutically acceptable salt thereof is an enhanced dose intensity.

In yet another embodiment of the method, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight.

In another preferred embodiment of the method, the therapeutically effective amount of enzastaurin (LY317615), LY326020, or a pharmaceutically acceptable salt thereof is in a range from about 250 mg to about 1000 mg per day. More preferably, the therapeutically effective amount of enzastaurin (LY317615), LY326020, or a pharmaceutically acceptable salt thereof is about 500 mg per day, about 700 mg per day, or about 900 mg per day. In a preferred embodiment of the method, the therapeutic components are administered daily in a 6 week cycle.

In one preferred embodiment, the glioma is an anaplastic astrocytoma. In another preferred embodiment, the glioma is a glioblastoma multiforme.

### BRIEF DESCRIPTION OF THE DRAWINGS

FIG. 1 illustrates mean tumor growth curves of U87MG (glioblastoma) xenograft tumors in nude mice dosed with control or various amounts of TMZ, enzastaurin, or a combination of both TMZ and enzastaurin. Dosing Regimens 1-10 (detailed in Table 1) are summarized below:
control (saline 10% DMSO qd, ip Day 1-5 +10% acacia qd, po
5 mpk (mg per kg), 10 mpk, or 20 mpk TMZ qd, ip Day 1-5
37.5 mpk or 75 mpk enzastaurin qd, po
10 mpk TMZ qd, ip Day 1-5 in combination with 37.5 or 75 mpk enzastaurin qd, po
20 mpk TMZ qd, ip Day 1-5 in combination with 37.5 or 75 mpk enzastaurin qd, po

FIG. 2 illustrates U87MG (glioblastoma) xenograft tumor size (mm³) in nude mice from Dosing Regimen 1-10 on Day 17 post inoculation as well as SEM, % inhibition, and % regression.

FIG. 3 illustrates the % body weight change from Day 7 to Day 17 post inoculation in nude mice with the U87MG (glioblastoma) xenograft tumors treated with Dosing Regimen 1-10.

### DETAILED DESCRIPTION OF THE INVENTION

As used herein, the following terms shall have the definitions set forth below.

As used herein, the phrase “selective PKC inhibitor” means an agent that inhibits ATP dependent signaling from a PKC isozyme (Le., conventional (e.g., α, β1, β2, γ); novel (e.g., δ, ε, η, θ, μ), atypical (e.g., ζ, λ), or a PKC-related kinase (e.g., PRKs 1, 2, and 3)) with more than about 10-fold greater potency than from one or more other ATP dependent kinases (e.g., protein kinase A, calcium calmodulin, casein kinase, src tyrosine kinase). Similarly, the phrase “selective conventional PKC inhibitor” refers to an agent that inhibits ATP dependent signaling from one or more conventional PKC isozymes (e.g., α, β1, β2, γ) with more than about 5-fold greater potency than from one or more non-conventional PKC isozymes. Similarly, the phrase “selective PKC alpha inhibitor” refers to an agent that inhibits ATP dependent signaling from PKC alpha isozyme with more than about 5-fold greater potency than from one or more other PKC isozymes. Similarly, the phrase “selective PKC beta inhibitor” refers to an agent that inhibits ATP dependent signaling from PKC beta isozyme with more than about 5-fold greater potency than from one or more other PKC isozymes. Likewise, the phrase “selective PKC beta-2 inhibitor” refers to an agent that inhibits ATP dependent signaling from PKC beta-2 with more than about 5-fold greater potency, than from one or more other PKC isozymes including PKC beta-1. Exemplary ATP dependent assays to determine the selectivity of agents as detailed above can be found in Jirousek et al., J Med Chem, 39:2664-2671 (1996) the entire disclosure of which is incorporated herein by reference. More specifically, Jirousek et al., J Med Chem, 39:2664-2671 (1996) describe a calcium calnodulin dependent protein kinase assay, a casein protein kinase II assay, a cAMP dependent PKCatalytic subunit assay, and a PKC enzyme assay. As used herein, the phrase “therapeutically effective amount” with respect to TMZ or a PKC inhibitor means an amount which provides a therapeutic benefit in the treatment or management of the referenced cell proliferative disorder (e.g., glioma, etc.). It is believed that such a combination will be more effective than treatment with either therapy alone. In addition, it is believed that the present formulations, kits, and methods will permit a lower dose of one or more pharmaceutically active agents to be administered, than would otherwise be required, to achieve a therapeutic effect thereby reducing adverse effects associated with the dosage administered.

As used herein the phrase “pharmaceutically acceptable salt” refers to a non-toxic salt prepared from a pharmaceutically acceptable acid or base (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine.

As used herein, the phrase “cell proliferative disorder” refers to a neoplasm. That is, a new, abnormal growth of cells or a growth of abnormal cells which reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is cancerous, and/or metastastic, Le., invades contiguous tissue or is no longer under normal cellular growth control. In preferred embodiments, the formulations, kits, and methods of the invention are used to treat cell proliferative disorders including but not limited to glioma, melanoma, prostate, lung cancer, breast cancer, ovarian, testicular cancer, gastric cancer, liver, kidney, spleen, bladder, colorectal and/or colon cancer, head and neck, carcinoma, sarcoma, lymphoma, leukemia or mycosis fungoides. In more preferred embodiments, the formulations, kits, and methods of the invention are used to treat glioma, melanoma, lung cancer, lymphoma, colorectal and/or colon cancer, head and neck or ovarian cancer. In a most preferred embodiment, the cell proliferative disorder is glioma.

As used herein the term “standard dose intensity” of TMZ means a 5/28 dosing regimen, with a dosing schedule of 150-200 mg/m² of TMZ per day, administered for 5 days in a 28 day cycle for a maximal total dose of 1000 mg/m²/4 weeks. This dosing regimen provides a “dose intensity” of 1.0.

As used herein the term “enhanced dose intensity” of TMZ means a dosing regimen and/or dosing schedule which provides a dose intensity of TMZ, which is 1.4-4.2, preferably 1.4-2.8, more preferably 1.8-2.8 times more intense (compared with the standard dose intensity). See, U.S. Patent Application Publication No. US 2006/0100188, Tables 1 and 2 at pages 2 and 3 for illustrative dosing regimens using enhanced dosing intensities, the entirety of which is hereby incorporated by reference.

As used herein, the term “treating” is intended to mean mitigating or alleviating a cell proliferative disorder (e.g., glioma, etc.) in a mammal such as a human.

As used herein the term “capsule” refers to a special container or enclosure made of methyl cellulose, polyvinyl alcohols, or denatured gelatins or starch for holding or containing a composition comprising a formulation of the present invention and a carrier. Hard shell capsules are typically made of blends of relatively high gel strength bone and pork skin gelatins. The capsule itself may contain small amounts of dyes, opaquing agents, plasticizers, and preservatives.

As used herein the term “tablet” refers to a compressed or molded solid containing a composition comprising a formulation of the present invention and a carrier with suitable diluents. The tablet can be prepared by compression of mixtures or granulations obtained by wet granulation, dry granulation or by compaction.

As used herein the phrase “oral gel” refers to a composition comprising a formulation of the present invention and a carrier dispersed or solubilized in a hydrophilic semi-solid matrix.

As used herein the phrase “orally consumable film” refers to a composition comprising a formulation of the present invention and an edible film carrier.

As used herein the phrase “powders for constitution” refers to powder blends containing a composition comprising a formulation of the present invention and a carrier with suitable diluents which can be suspended in water or juices.

As used herein the term “diluent” refers to a substance that usually makes up the major portion of the composition. Suitable diluents include sugars such as lactose, sucrose, mannitol, and sorbitol; starches derived from wheat, corn rice, and potato; and celluloses such as microcrystalline cellulose. The amount of diluent in the composition can range from about 10% to about 90% by weight of the total composition, preferably from about 25% to about 75%, more preferably from about 30% to about 60% by weight, even more preferably from about 12% to about 60%.

As used herein the term “disintegrant” refers to a substance added to the composition to help it break apart (disintegrate) and release the medicinal agent(s). Suitable disintegrants include starches; “cold water soluble” modified starches such as sodium carboxymethyl starch; natural and synthetic gums such as locust bean, karaya, guar, tragacanth, and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2% to about 15% by weight of the composition, more preferably from about 4% to about 10% by weight.

As used herein the term “binder” refers to a substance that binds or “glues” powders together and makes them cohesive by forming granules, thus serving as the “adhesive” in the composition. Binders add cohesive strength already available in the diluent or bulking agent. Suitable binders include sugars such as sucrose; starches derived from wheat, corn rice, and potato; natural gums such as acacia, gelatin, and tragacanth; derivatives of seaweed such as alginic acid, sodium alginate, and ammonium calcium alginate; cellulosic materials such as methylcellulose, sodium carboxymethylcellulose, and hydroxypropylmethylcellulose; polyvinylpyrrolidinone; and inorganics such as magnesium aluminum silicate. The amount of binder in the composition can range from about 2% to about 20% by weight of the composition, more preferably from about 3% to about 10% by weight, even more preferably from about 3% to about 6% by weight.

As used herein the term “lubricant” refers to a substance added to the composition to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols, and d'l-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

As used herein the term “glidant” refers to a substance that prevents caking and improves the flow characteristics of granulations, so that flow is smooth and uniform. Suitable glidants include silicon dioxide and talc. The amount of glidant in the composition can range from about 0.1% to about 5% by weight of the total composition, preferably from about 0.5% to about 2% by weight.

As used herein the phrase “coloring agent” refers to a substance that provides coloration to the composition. Such substances can include food grade dyes and food grade dyes adsorbed onto a suitable adsorbent such as clay or aluminum oxide. The amount of the coloring agent can vary from about 0.1% to about 5% by weight of the composition, preferably from about 0.1% to about 1%.

In one embodiment, the formulations and kits of the present invention are for oral administration. For oral preparations, a pharmaceutically acceptable carrier (which includes diluents, excipients, or carrier materials) is also present in the formulation. The carrier is suitably selected with respect to the intended form of administration, Le., oral tablets, capsules (either solid-filled, semi-solid filled, or liquid filled), powders for constitution, oral gels, orally consumable films, elixirs, syrups, suspensions, and the like, and consistent with conventional pharmaceutical practices. For example, for oral administration in the form of tablets or capsules, the pharmaceutically active agents may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms), and the like. Moreover, when desired or needed, suitable binders, lubricants, disintegrants, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol, and waxes. Suitable lubricants include boric acid, sodium benzoate, sodium acetate, sodium chloride, and the like. Suitable disintegrants include starch, methylcellulose, guar gum, and the like. Suitable disinfectants include benzalkonium chloride and the like. Sweetening and flavoring agents and preservatives may also be included where appropriate.

Additionally, the formulations and kits of the present invention may be formulated in sustained release form to provide the rate controlled release of any one or more of the pharmaceutically active agents to optimize the therapeutic effects. Suitable compositions for sustained release include layered tablets (e.g., containing layers of varying disintegration rates or controlled release polymeric matrices impregnated with the medicinal agents) that are shaped in tablet form or capsules containing such impregnated or encapsulated porous polymeric matrices.

Conventional methods for preparing tablets are known. Such methods include dry methods such as direct compression and compression of granulation produced by compaction, wet methods, or other special procedures.

In another embodiment, the formulations and kits of the present invention are for parenteral administration, for example, intravenous, intratumoral, subcutaneous, or intramuscular administration.

Thus, to prepare an aqueous solution for parenteral injection, it is possible to use a co-solvent, e.g., an alcohol such as ethanol or a glycol such as polyethylene glycol or propylene glycol, or glycerin, and optionally, a hydrophilic surfactant such as Tween® 80. An oily solution injectable intramuscularly can be prepared, e.g., by solubilizing the active principle with a triglyceride or a glycerol ester. The substantially non-aqueous carrier (excipient) can be any substance that is biocompatible and liquid or soft enough at body temperature. The carrier is usually hydrophobic and commonly organic, e.g., an oil or fat of vegetable, animal, mineral or synthetic origin or derivation. Preferably, but not necessarily, the carrier includes at least one chemical moiety of the kind that typifies “fatty” compounds, e.g., fatty acids, alcohols, esters, etc., Le., a hydrocarbon chain, an ester linkage, or both. “Fatty” acids in this context include acetic, propionic and butyric acids, through straight- or branched-chain organic acids containing up to 30 or more carbon atoms. Preferably, the carrier is immiscible in water and/or soluble in the substances commonly known as fat solvents. The carrier can correspond to a reaction product of such a “fatty” compound or compounds with a hydroxy compound, e.g., a mono-hydric, di-hydric, trihydric or other polyhydric alcohol, e.g., glycerol, propanediol, lauryl alcohol, polyethylene or -propylene glycol, etc. These compounds include the fat-soluble vitamins, e.g., tocopherols and their esters, e.g., acetates sometimes produced to stabilize tocopherols. Sometimes, for economic reasons, the carrier may preferably comprise a natural, unmodified vegetable oil such as sesame oil, soybean oil, peanut oil, palm oil, or an unmodified fat. Alternatively the vegetable oil or fat may be modified by hydrogenation or other chemical means which is compatible with the present invention. The appropriate use of hydrophobic substances prepared by synthetic means is also envisioned.

Pharmaceutical compositions suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g., human serum albumin), toxicity agents (e.g., NaCl), preservatives (e.g., thimerosol, cresol or benylalcohol), and surfactants (e.g., Tween or polysorabates) in sterile water for injection.

Typical suitable syringes include systems comprising a prefilled vial attached to a pen-type syringe such as the NOVOLET Novo Pen available from Novo Nordisk, as well as prefilled, pen-type syringes which allow easy self-injection by the user. Other syringe systems include a pen-type syringe comprising a glass cartridge containing a diluent and lyophilized powder in a separate compartment.

The present invention provides TMZ or a pharmaceutically acceptable salt thereof in combination with a selective PKC inhibitor, preferably an isozyme-specific PKC inhibitor. In one embodiment, a selective PKC beta inhibitor.

TMZ is an alkylating agent available under the trademark Temodar® from Schering Corporation (Kenilworth, N.J.). TMZ is also known as 3,4-dihydro-3-methyl-4-oxoimidazo[5,1-d]-as-tetazine-8-carboxamide. See U.S. Pat. No. 5,260,291, incorporated herein by reference in its entirety. TMZ is currently approved in the United States for the treatment of adult patients with high grade gliomas that include newly diagnosed glioblastoma multiforme and refractory anaplastic astrocytoma (Le., patients at first relapse who have experienced disease progression on a drug regimen containing a nitrosourea and procarbazine). TMZ is currently approved in Europe for the treatment of patients with malignant glioma, such as glioblastoma multiforme or anaplastic astrocytoma showing recurrence or progression after standard therapy. TMZ may be administered as an oral or intravenous dose in the range of about 150 to about 200 mg/m² per day for 5 days in a 28-day treatment cycle. In one embodiment, the therapeutically effective amount of TMZ or a pharmaceutically acceptable salt thereof is either a standard or enhanced dose intensity of TMZ based upon the methylation state of the O⁶-methylguanine-DNA methyltransferase (MGMT) gene in a sample obtained from the patient. If the gene (e.g., the promoter region) encoding MGMT in a sample from the patient is methylated, a standard dose intensity of TMZ is administered; however, if the gene encoding MGMT is not methylated (Le., below the level of detection), an enhanced dose intensity of TMZ is administered to the patient. See, U.S. Patent Application Publication No. US 2006/0100188, in particular, exemplary enhanced dose intensities for TMZ are provided in Tables 1 and 2; methods to assess whether or not the MGMT gene is methylated are provided on pages 15-20; and the term “sample” is defined on page 13; all disclosures incorporated herein in their entireties by reference herein.

Generally, an amount of selective PKC inhibitor to be administered in combination with TMZ is decided on a case by case basis by the attending physician. As a guideline, the extent of the cell proliferative disorder, the body weight, and the age of the patient will be considered, among other factors, when setting an appropriate dose.

Generally, a suitable dose of selective PKC inhibitor is one that results in a concentration of the selective PKC inhibitor at the site of tumor cells in the range of 0.5 nM to 200 μM, and more usually from 20 nM to 10 μM. It is expected that serum concentrations of 100 nM to 5 μM should be sufficient in most circumstances. To obtain these treatment concentrations of selective PKC inhibitor, a patient in need of treatment likely will be administered between about 0.1 mg to about 14 mg per day per kg of bodyweight. For example, in one embodiment, the therapeutically effective amount of ruboxistaurin (LY333531), N-desmethyl LY333531, or a pharmaceutically acceptable salt thereof is in a range from about 0.1 mg per day per kg of body weight to about 1.5 mg per day per kg of body weight. More preferably, the therapeutically effective amount of ruboxistaurin (LY333531), N-desmethyl LY333531, or a pharmaceutically acceptable salt thereof is about 1.0 mg per day per kg of body weight. In another embodiment, the therapeutically effective amount of enzastaurin (LY317615), LY326020, or a pharmaceutically acceptable salt thereof is in a range from about 3.5 mg per day per kg of body weight to about 14 mg per day per kg of body weight (e.g., in a range from about 250 mg to about 1000 mg per day, based on an average body weight of 70 kg). More preferably, the therapeutically effective amount of enzastaurin (LY317615), LY326020, or a pharmaceutically acceptable salt thereof is about 500 mg per day, about 700 mg per day, or about 900 mg per day.

As noted above, the above amounts may vary on a case-by-case basis.

Exemplary selective PKC inhibitors are described in the art, e.g., Mackay and Twelves, Targeted Therapies in Oncology, 4(1):7-10 (2004); Rocha et al., The Oncologist, 7:17-33 (2002); Way et al., Trends Pharmacol Sci 21(5):181-187 (2000); U.S. Pat. Nos. 5,621,101, 5,621,098, 5,616,577, 5,578,590, 5,552,396, 5,545,636, 5,491,242, 5,488,167, 5,481,003, 5,461,146, 5,270,310, 5,216,014, 5,204,370, 5,141,957, 5,057,614, 4,990,519, and 4,937,232. Notably, staurosporine, a potent PKC inhibitor, has served as a lead compound from which many novel PKC inhibitors have been developed with improved selectivity for PKC as well as for greater selectivity with respect to PKC isozymes. Such derivatives include UCN01, GÖ6976, ruboxistaurin (LY333531), LY379196, and enzastaurin (LY317615).

UCN01 (7-hydroxystaurosporine), and related analogues N-benzoyl-staurosporine and CGP 41251 are described in Rocha et al., The Oncologist, 7:17-33 (2002). In one preferred embodiment, UCNO1 or a pharmaceutically acceptable salt thereof is administered parenterally, more preferably, intravenously.

GÖ6976 (12H-indolo (2,3-α)pyrrolo-3,4-t)carbazole-12-propanenitrile,5,6,7,13-tetrahydro-13-methyl-5-oxo[MESH]) is available from Biomol (Plymouth Meeting, Pa.). GÖ6976 is described in Hai et al., Experimental Cell Research, 280:64-74 (2002). In one preferred embodiment, GÖ6976 or a pharmaceutically acceptable salt thereof is administered parenterally, more preferably, intravenously.

Ruboxistaurin (LY33353 1; (S)-13-[(dimethylamino)methyl]-10,11,14,15-tetrahydro-4,9:16,21-dimetheno-1H,13H-dibenzo[e,k]pyrrolo[3,4-h][1,4,13]oxadiazacyclohexadecene-1,3(2H)-dione) and related analogues (including the active metabolite of LY33353 1, N-desmethyl LY33353 1) are described in Jirousek et al., J Med Chem, 39:2664-2671 (1996). N-desmethyl LY333531 is further described in Ring et al., Drug Metabolism and Disposition, 30(9):957-961 (2002). In one embodiment, the therapeutically effective amount of ruboxistaurin (LY33353 1) or a pharmaceutically acceptable salt thereof is in a range from about 1 mg to about 500 mg per day, more preferably, from about 5 mg to about 300 mg per day. Exemplary formulations are provided in Health et al., U.S. Pat. No. 5,552,396. Exemplary dosages are described in Jirousek et Cd., U.S. Patent Publication US 2001/0001791. In one preferred embodiment, ruboxistaurin (LY33353 1) or a pharmaceutically acceptable salt thereof is administered orally.

LY379196, an analog of ruboxistaurin (LY333531) is described in Slosberg et al., Mol Carcinog, 27(3):166-176 (2000)] and Heath, U.S. Pat. No. 5,552,396). In one preferred embodiment, LY379196 or a pharmaceutically acceptable salt thereof is administered orally. Enzastaurin (LY317615) is described in Mackay and Twelves, Targeted Therapies in Oncology, 4(1):7-10 (2004) and Graff et al., Cancer Res, 65(16):7462-7469 (2005). LY326020, the primary active metabolite of enzastaurin (LY317615) is described in Herbst et al., 2002 American Society of Clinical Oncology (ASCO) Annual Meeting, Abstract #326. In one embodiment, the therapeutically effective amount of enzastaurin (LY317615), LY326020, or a pharmaceutically acceptable salt thereof is in a range from about 250 mg to about 1000 mg per day for a 6 week cycle. Preferably, about 500 mg, about 700 mg, or about 900 mg per day on a 6 week cycle. In one preferred embodiment, enzastaurin (LY317615), LY326020, or a pharmaceutically acceptable salt thereof is administered orally.

Bryostatin 1, a macrocyclic lactone originally isolated from marine bryophyte Bugula neritina is described in Rocha et al., The Oncologist, 7:17-33 (2002). In one preferred embodiment, bryostatin 1 or a pharmaceutically acceptable salt thereof is administered parenterally, more preferably, intravenously.

Tamoxifen citrate, an anti-estrogen agent, is available, for example, under the trademark Nolvadex® from AstraZeneca Pharmaceuticals (Wilmington, Del.). Tamoxifen citrate is also known as (Z)2-[4-(1,2-diphenyl-1-butenyl)phenoxy]-N,N-dimethylethanamine 2-hydroxy-1,2,3-propranetricarboxylate (1:1). See U.S. Pat. No. 5,470,883, incorporated herein by reference in its entirety. Tamoxifen citrate is currently approved in the United States for the treatment of breast cancer. In one embodiment, the therapeutically effective amount of tamoxifen or a pharmaceutically acceptable salt thereof is in a range from about 10 mg to about 1000 mg per day. Preferably, about 20 mg, about 40 mg, or about 90 mg per day. In another embodiment, the therapeutically effective amount of tamoxifen or a pharmaceutically acceptable salt thereof is about 2 mg per kg per day. In one preferred embodiment, tamoxifen or a pharmaceutically acceptable salt thereof is administered orally.

Antisense oligonucleotide selective PKC inhibitors include ISIS 3521 (LY900003; Affinitak) and ISIS 9606. Both of these antisense oligonucleotides are selective PKC alpha inhibitors. The sequences for these antisense oligonucleotides are as follows: ISIS 3521 (LY900003; Affinitak) GTT CTC GCT GGT GAG TTT CA (SEQ ID NO: 1) and ISIS 9606 GTT CTC GCT GGT GAG TTT CA (SEQ ID NO: 2) (see, e.g., McKay et al., Cancer Res, 56(15):3499-3507 (1996) and Levesque et al., Mol Pharmacol, 51(2):209-216 (1997)). In one embodiment, the therapeutically effective amount of ISIS 3521 (LY900003; Affinitak) is in a range from about 2.0 mg/kg per day to about 3.0 mg/kg per day given over 21 days followed by a 7-day rest period. In another embodiment, the therapeutically effective amount of ISIS 3521 (LY900003; Affinitak) administered is in a range from about 0.15 mg/kg/day to about 6.0 mg/kg/day) 3 times per week. In yet another embodiment, the therapeutically effective amount of ISIS 9606 administered results in a concentration of about 150 nM at the site of tumor cells. In one preferred embodiment, such antisense oligonucleotides are administered parenterally, preferably intravenously. Lastly, exemplary peptide translocation inhibitors of PKC are described in Way et al., Trends Pharmacol Sci, 21(5): 181-187 (2000).

The following human glioblastoma xenograft model may be employed to ascertain the efficacy of the formulations and methods described herein. Human glioblastoma cell U87MG are inoculated subcutaneously into female nude mice (age 4-6 wks). Xenograft tumor growth is followed by measuring the tumor size using a caliper. Once tumor size reaches about 100 mm3 (average), mice carrying the xenograft tumors are grouped and treated with different doses of the combination of TMZ and a PKC inhibitor (e.g., enzastaurin). Likewise, human glioblastoma cell U373 may be used to establish a xenograft model.

### EXAMPLE

Efficacy of TMZ and/or Enzastaurin in U87MG Glioblastoma Xenografts

The combination of TMZ and a PKC inhibitor (e.g., enzastaurin) for treating a cell proliferative disorder was examined using a glioblastoma xenograft model. In particular, the glioblastoma model U87MG was used to evaluate the efficacy of PKC inhibitor enzataurin as a single agent compared to TMZ (Temodar®), a chemotherapeutic drug clinically approved for treating brain tumor, as a single agent. In addition, this study looked at the efficacy of the combination of TMZ with enzastaurin in comparison to the efficacy with either agent alone.

In brief, nude mice were inoculated with U87-MG glioblastoma cells and the resultant tumors treated with control, or various concentrations of TMZ, enzastaurin, or a combination of both TMZ and enzastaurin. More specifically, 150 female nude mice (strain NU/NU), aged 5-7 weeks old were purchased from Charles River Laboratory. Four million glioblastoma cells U87-MG were mixed 1:1 (volume:volume) with Matrigel (Cat. #354234, BD Biosciences) on ice and mixtures were inoculated subcutaneously to the flank of each mouse. Dosing was initiated when the tumors reached an average size of 90 mm³. The dosing volume was 0.2 mL. Tumor size and body weight was measured two to three times per week. Table 1 below displays the 10 different dosing regimens. Note that there were 10 nude mice per dosing regimen.

TABLE 1

| Dosing | | | |
|---|---|---|---|
| Regimen | Dose 1 (qd, ip, Day 1-5) | Dose 2 (qd, po) | |
| 1 | Saline 10% DMSO | 10% | Acacia |
| 2 | 5 mpk TMZ | — | |
| 3 | 10 mpk TMZ | — | |
| 4 | 20 mpk TMZ | — | |
| 5 | — | 37.5 | mpk enzastaurin |
| 6 | — | 75 | mpk enzastaurin |
| 7 | 10 mpk TMZ | 37.5 | mpk enzastaurin |
| 8 | 20 mpk TMZ | 37.5 | mpk enzastaurin |
| 9 | 10 mpk TMZ | 75 | mpk enzastaurin |
| 10 | 20 mpk TMZ | 75 | mpk enzastaurin |

The mean tumor growth curves of U87MG (glioblastoma) xenograft tumors from Dosing Regimen 1-10 illustrate that TMZ alone or in combination with enzastaurin is more effective than enzastaurin alone at decreasing tumor growth in U87MG glioblastoma xenografts (FIG. 1).

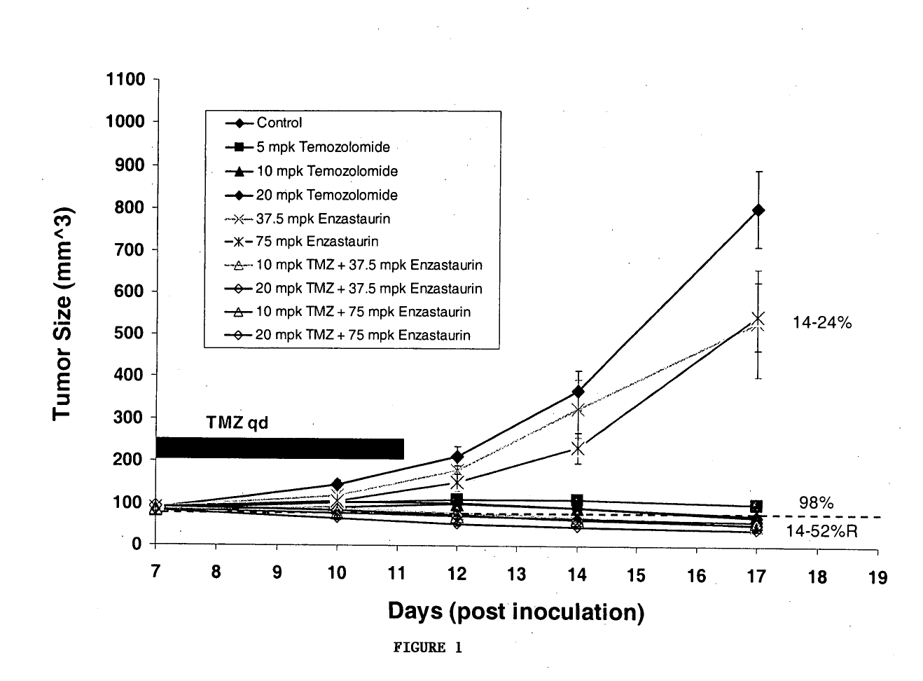

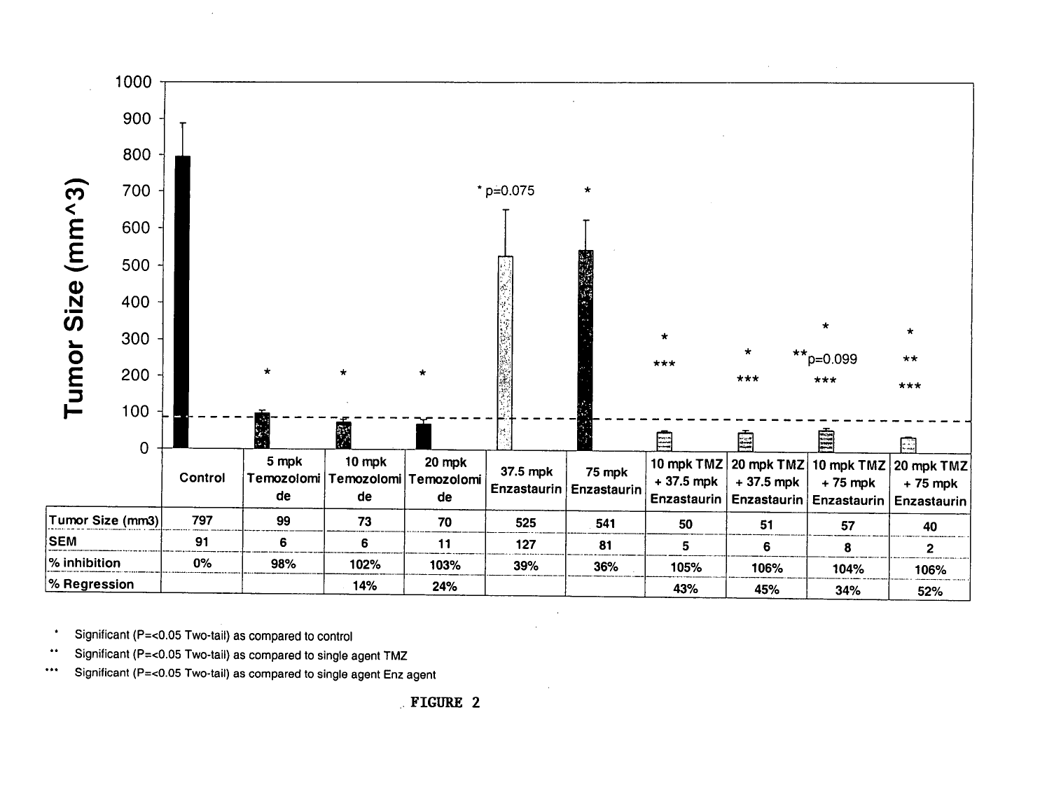

Specifically, TMZ alone inhibited the U87MG glioblastoma xenograft tumor growth by 98%, 102% and 103%, at 5, 10 and 20 mpk dose levels, respectively (see, FIG. 1 and FIG. 2). In contrast, PKC inhibitor enzastaurin had little effect on tumor growth when used as single agent (see, FIG. 1 and FIG. 2).

Moreover, TMZ in combination with enzastaurin was more effective than TMZ alone. In particular, on Day 17 post inoculation (Le., day 10 post dosing initiation), a dose-dependent decrease in tumor growth and increase of tumor regression was observed when TMZ is combined with enzastaurin (see, FIG. 2).

Notably, the highest combination dose (Le., 20 mpk TMZ plus 75 mpk Enzastaurin) resulted in 52% tumor regression (compared to its initial size when dosing initiated on day 7 post inoculation). This was statistically better than either TMZ or Enzastaurin used alone (p<0.05).

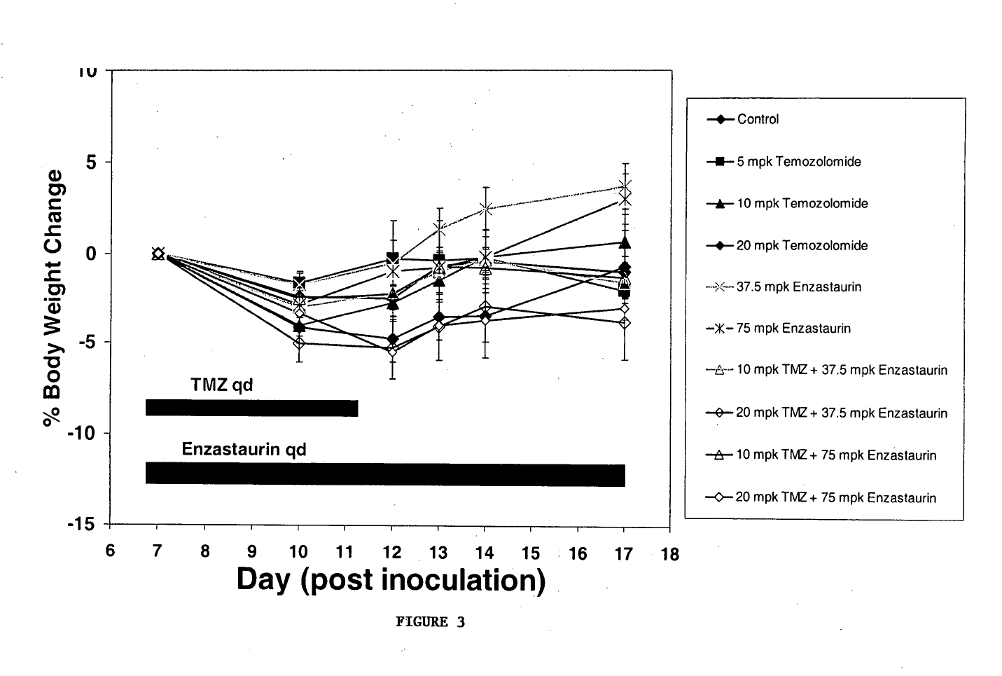

It should also be noted that mice tolerated the dosage regimens of TMZ and Enzastaurin well, exhibiting no more than 5% body weight loss (see, FIG. 3). In fact, the combination of TMZ with Enzastaurin does not adversely affect the body weight any more than treatment with either agent alone.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed:

1. A formulation comprising a therapeutically effective amount of temozolomide (TMZ) or a pharmaceutically acceptable salt thereof in combination with a protein kinase C (PKC) inhibitor.

2. A formulation comprising a therapeutically effective amount of TMZ or a pharmaceutically acceptable salt thereof in combination with a selective PKC beta inhibitor.

3. The formulation of claim 2 wherein the selective PKC beta inhibitor is a selective PKC beta-2 inhibitor.

4. The formulation of claim 2 wherein the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

5. The formulation of claim 2 wherein the selective PKC beta inhibitor is ruboxistaurin (LY333531), N-desmethyl LY333531, or a pharmaceutically acceptable salt thereof.

6. The formulation of claim 2 wherein the selective PKC beta inhibitor is N-desmethyl LY33353 1, or a pharmaceutically acceptable salt thereof.

7. The formulation of claim 2 wherein the selective PKC beta inhibitor is enzastaurin (LY317615), LY326020, or a pharmaceutically acceptable salt thereof.

8. The formulation of claim 2 wherein the selective PKC beta inhibitor is LY326020, or a pharmaceutically acceptable salt thereof.

9. The formulation of claim 2 wherein the therapeutically effective amount of TMZ or a pharmaceutically acceptable salt thereof is a standard dose intensity.

10. The formulation of claim 2 wherein the therapeutically effective amount of TMZ or a pharmaceutically acceptable salt thereof is an enhanced dose intensity.

11. The formulation of claim 5 wherein ruboxistaurin (LY333531), N-desmethyl LY333531, or a pharmaceutically acceptable salt thereof is in a range from about 0.1 mg per day per kg of body weight to about 1.5 mg per day per kg of body weight.

12. The formulation of claim 5 wherein ruboxistaurin (LY333531), N-desmethyl LY333531, or a pharmaceutically acceptable salt thereof is about 1.0 mg per day per kg of body weight.

13. The formulation of claim 7 wherein enzastaurin (LY317615), LY326020, or a pharmaceutically acceptable salt thereof is in a range from about 250 mg to about 1000 mg per day.

14. The formulation of claim 7 wherein enzastaurin (LY317615), LY326020, or a pharmaceutically acceptable salt thereof is about 500 mg per day.

15. The formulation of claim 7 wherein enzastaurin (LY317615), LY326020, or a pharmaceutically acceptable salt thereof is about 700 mg per day.

16. The formulation of claim 7 wherein enzastaurin (LY317615), LY326020, or a pharmaceutically acceptable salt thereof is about 900 mg per day.

17. A kit comprising:
a first container having a therapeutically effective amount of TMZ or a pharmaceutically acceptable salt thereof;
a second container having a therapeutically effective amount of a selective PKC beta inhibitor; and
instructions for use to treat a cell proliferative disorder.

18. The kit of claim 17 wherein the selective PKC beta inhibitor is a selective PKC beta-2 inhibitor.

19. The kit of claim 17 wherein the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

20. The kit of claim 17 wherein the selective PKC beta inhibitor is ruboxistaurin (LY333531), N-desmethyl LY333531, or a pharmaceutically acceptable salt thereof.

21. The kit of claim 17 wherein the selective PKC beta inhibitor is N-desmethyl LY33353 1, or a pharmaceutically acceptable salt thereof.

22. The kit of claim 17 wherein the selective PKC beta inhibitor is enzastaurin (LY317615), LY326020, or a pharmaceutically acceptable salt thereof.

23. The kit of claim 17 wherein the selective PKC beta inhibitor is LY326020, or a pharmaceutically acceptable salt thereof.

24. The kit of claim 17 wherein the therapeutically effective amount of TMZ or a pharmaceutically acceptable salt thereof is a standard dose intensity.

25. The kit of claim 17 wherein the therapeutically effective amount of TMZ or a pharmaceutically acceptable salt thereof is an enhanced dose intensity.

26. The kit of claim 20 wherein ruboxistaurin (LY333531), N-desmethyl LY333531, or a pharmaceutically acceptable salt thereof is in a range from about 0.1 mg per day per kg of body weight to about 1.5 mg per day per kg of body weight.

27. The kit of claim 20 wherein ruboxistaurin (LY333531), N-desmethyl LY333531, or a pharmaceutically acceptable salt thereof is about 1.0 mg per day per kg of body weight.

28. The kit of claim 22 wherein enzastaurin (LY317615), LY326020, or a pharmaceutically acceptable salt thereof is in a range from about 250 mg to about 1000 mg per day.

29. The kit of claim 22 wherein enzastaurin (LY317615), LY326020, or a pharmaceutically acceptable salt thereof is about 500 mg per day.

30. The kit of claim 22 wherein enzastaurin (LY317615), LY326020, or a pharmaceutically acceptable salt thereof is about 700 mg per day.

31. The kit of claim 22 wherein enzastaurin (LY317615), LY326020, or a pharmaceutically acceptable salt thereof is about 900 mg per day.

32. The kit of claim 17 wherein the therapeutically effective amount of TMZ or a pharmaceutically acceptable salt thereof is administered together in time as the therapeutically effective amount of the selective PKC beta inhibitor.

33. The kit of claim 17 wherein the therapeutically effective amount of TMZ or a pharmaceutically acceptable salt thereof is administered separately in time as the therapeutically effective amount of the selective PKC beta inhibitor.

34. A method for treating a glioma in a patient suffering there from comprising administering a therapeutically effective amount of TMZ or a pharmaceutically acceptable salt thereof in combination with a selective PKC beta inhibitor.

35. The method of claim 34 wherein the selective PKC beta inhibitor is a selective PKC beta-2 inhibitor.

36. The method of claim 34 wherein the selective PKC beta inhibitor is ruboxistaurin (LY333531), N-desmethyl LY333531, LY379196, enzastaurin (LY317615), LY326020, or a pharmaceutically acceptable salt thereof, or a combination of two or more thereof.

37. The method of claim 34 wherein the selective PKC beta inhibitor is ruboxistaurin (LY333531), N-desmethyl LY333531, or a pharmaceutically acceptable salt thereof.

38. The method of claim 34 wherein the selective PKC beta inhibitor is N-desmethyl LY333531, or a pharmaceutically acceptable salt thereof.

39. The method of claim 34 wherein the selective PKC beta inhibitor is enzastaurin (LY317615), LY326020, or a pharmaceutically acceptable salt thereof.

40. The method of claim 34 wherein the selective PKC beta inhibitor is LY326020, or a pharmaceutically acceptable salt thereof.


SEQUENCE LISTING (2 sequences)

SEQ ID NO: 1 - DNA, 20 bp, Artificial
  ISIS 3521 (LY900003; Affinitak); antisense oligonucleotide
gttctcgctggtgagtttca

SEQ ID NO: 2 - DNA, 20 bp, Artificial
  ISIS 9606; antisense oligonucleotide
  modified_base (1)..(1): 2′-O-methoxyethyl nucleotide
  modified_base (2)..(2): 2′-O-methoxyethyl nucleotide
  modified_base (3)..(3): 2′-O-methoxyethyl nucleotide
  modified_base (4)..(4): 2′-O-methoxyethyl nucleotide
  modified_base (5)..(5): 2′-O-methoxyethyl nucleotide
  modified_base (6)..(6): 2′-O-methoxyethyl nucleotide
  modified_base (15)..(15): 2′-O-methoxyethyl nucleotide
  modified_base (16)..(16): 2′-O-methoxyethyl nucleotide
  modified_base (17)..(17): 2′-O-methoxyethyl nucleotide
  modified_base (18)..(18): 2′-O-methoxyethyl nucleotide
  modified_base (19)..(19): 2′-O-methoxyethyl nucleotide
  modified_base (20)..(20): 2′-O-methoxyethyl nucleotide
gttctcgctggtgagtttca